(19) United States
(12) Patent Application Publication
(10) Pub. No.: US 2017/0216213 A1
(43) Pub. Date: Aug. 3, 2017

(54) DIRECTLY COMPRESSIBLE POLYVINYL ALCOHOLS

(71) Applicant: Merck Patent GmbH, Darmstadt (DE)

(72) Inventors: Roberto OGNIBENE, Darmstadt (DE); Finn BAUER, Bensheim (DE); Thorsten WEDEL, Stockstadt/Rhein (DE); Guenter MODDELMOG, Reinheim (DE)

(73) Assignee: Merck Patent GmbH, Darmstadt (DE)

(21) Appl. No.: 15/329,421

(22) PCT Filed: Jul. 3, 2015
(86) PCT No.: PCT/EP2015/001355

§ 371 Date: Jan. 26, 2017

(30) Foreign Application Priority Data
Jul. 30, 2014 (EP) 14002666.7

(51) Int. Cl. A61K 9/20; C08L 1/04; C08L 29/04; A61K 31/375

(52) U.S. Cl. CPC A61K 9/2027; A61K 9/2095; A61K 9/2054; A61K 31/375; C08L 1/04; C08L 29/04; C08L 2203/02

(57) ABSTRACT

The present invention relates to directly compressible co-mixtures for the production of tablets having delayed release of active compound which comprise polyvinyl alcohols (PVAs) and microcrystalline celluloses (MCCs). The invention also relates to a process for the preparation of corresponding directly compressible co-mixture

The present invention relates to directly compressible co-mixtures for the production of tablets having delayed release of active compound which comprise polyvinyl alcohols (PVAs) and microcrystalline celluloses (MCCs). The invention also relates to a process for the preparation of corresponding directly compressible co-mixtures.

### PRIOR ART

Polyvinyl alcohol (PVA) is a synthetic, flexible polymer which is obtained by alkaline hydrolysis of polyvinyl acetate. Polyvinyl acetate is in turn obtained by free-radical polymerisation from vinyl acetate. Through different chain lengths and different degrees of hydrolysis of the polyvinyl acetates, polyvinyl alcohols (PVAs) having a very wide variety of physical properties can be obtained. The PVAs are employed, in particular, as film formers, adhesive gels and as viscosity modulator, in a multiplicity of areas of application, for example paints, papers, textiles, cosmetics, etc.

Of particular interest for the pharmaceutical industry is the use of PVAs in pharmaceutical preparations, such as, for example, in ophthalmic preparations, as film formers for coated tablets, as binders in granules or as coating component in plasters, and also in drug delivery systems. Of very particular interest is the use of various PVA grades in the formulation of solid oral pharmaceutical administration forms having extended release of active compound, for example in so-called “retard tablets”. Delayed release of active compound is achieved in polymer-containing pharmaceutical formulations of this type through the tablets not dissolving directly in the presence of liquid, such as in the mouth or gastrointestinal tract, but instead swelling and the active compound only being released little by little by diffusion.

Galenically modified tablets of this type enable the active compound to be released from the administration form in a controlled manner over an extended time in the body, in order thus to maintain a therapeutically effective blood level of the medicament over an extended period (several hours). The two essential advantages of such retarded formulations are—in contrast to tablets having immediate release of active compound after taking—firstly the avoidance of undesired, possibly also toxic blood/plasma levels of the API and also a reduction in the frequency with which the tablets are taken (for example only once/daily instead of 3 times/daily) and thus an improvement in so-called patient compliance together with an improved therapeutic result of the medicinal treatment.

Known polyvinyl alcohols which are specified for use in pharmaceutical formulations according to the various pharmacopoeias (Pharmacopoea Europaea, Ph. Eur.; United States Pharmacopoeia (USP), and the Japanese Pharmacopoeia (JP or JPE)), but cannot be tableted directly by the action of pressure or only under particular conditions. A particular problem in this connection thus consists in the production in a simple manner of tablets which principally consist of corresponding PVAs as active compound excipient in which the active compound is homogeneously distributed. Direct tabletability of PVA-containing formulations usually has to be achieved in the presence of relatively high proportions of further binders, such as lactose, and of lubricants and possibly further additives. Formulations in which PVAs are employed as active compound excipient are frequently prepared in the presence of aqueous or alcoholic solutions. For example, it is known to produce corresponding tablets having extended release of active compound by compressing the active compound and PVA in the presence of further additives after wet granulation. The latter is associated with the disadvantage that the requisite solvents have to be removed again with input of energy.

Object

As can be seen from the description above, swelling polymers, from which the active compound is released in a time-controlled manner via diffusion and erosion processes after moistening, for example, in the stomach and intestine and made available for resorption, are frequently employed in order to achieve the desired retardation effects. Known examples of such polymers are, in particular, modified celluloses, such as the hydroxypropylmethylcelluloses (HPMCs). However, the polyvinyl alcohols (PVAs), in particular, are also known for such retardation effects. PVAs are used if, for example, incompatibility reactions exist between active compound and HPMC or if the HPMC grades employed exhibit an unsatisfactory release profile of the active compound. For rapid tablet development with retardation effect, the pharmaceutical formulation scientist requires a swelling polymer which is directly compressible and nevertheless releases the active compound in a time-controlled manner. However, pulverulent PVAs are per se not directly compressible—they give tablets of unsatisfactory hardness which cannot be handled in pharmaceutical practice, since, for example, they have an undesired tendency to break or have excessively high abrasion.

The object of the present invention is therefore to provide directly compressible retardation matrices which make time-consuming granulation processes superfluous; i.e. steps which consist of moistening with granulation liquids, mechanical mixing in mixing systems or fluidised-bed equipment, and post-drying processes for the removal of the granulation liquids and sieving, so that time and energy can be saved, but also expensive and time-consuming investment in special granulation equipment. The object of the present invention is also to provide advantageous directly compressible retardation matrices of this type based on PVAs. The object of the present invention is, in addition, to provide a process by means of which PVAs, or commercially available PVA grades, can be converted into a directly compressible state.

### BRIEF DESCRIPTION OF THE INVENTION

The present invention provides the pharmaceutical formulation scientist with a directly compressible composition having extended release of active compound, comprising a co-mixture of polyvinyl alcohols (PVAs) and microcrystalline celluloses (MCCs). The present invention preferably relates to mixtures in which the polyvinyl alcohols (PVAs) and microcrystalline celluloses (MCCs) employed meet the requirements of the pharmacopoeias (Ph. Eur., USP and JPE. In accordance with the invention, corresponding directly compressible compositions may comprise polyvinyl alcohols (PVAs) of grades 18-88, 26-88 and 40-88 and all grades in between, including grade 28-99 in accordance with JPE and Ph. Eur. The object of the present invention is achieved, in particular, by directly compressible compositions comprising polyvinyl alcohols (PVAs) which conform to Ph. Eur. and which have been obtained by polymerisation of vinyl acetate and by subsequent partial or virtually complete hydrolysis of the polyvinyl acetate. Particularly suitable compositions are those which comprise polyvinyl alcohols (PVAs) which have been obtained by 85%-89% hydrolysis. Especially suitable are corresponding compositions which comprise polyvinyl alcohols (PVAs) which are water-swellable resins which, according to USP, are characterised by the formula

(C₂H₄O)ₙ

in which
n denotes an integer in the range from 500 to 5,000, and which have an average relative molecular weight in the range between 20,000 and
150,000 g/mol, which have a viscosity in accordance with Ph. Eur. in the range 3-70 mPa·s, (measured in a 4% solution at 20° C.) and which have an ester value of not greater than 280 mg of KOH/g (degree of hydrolysis >72.2 mol %).

Directly compressible compositions according to the invention having improved properties comprise the PVAs and MCCs described in a co-mixture in a ratio in the range 2:1 to 1:2, based on the weight, preferably in a ratio in the range from 2:1 to 1:1. After intensive mixing, the co-mixtures of PVA with MCCs found here have bulk densities in the range 0.40-0.48 g/ml with tapped densities in the range 0.55-0.63 g/ml.

In addition, the present invention also relates to an active compound-containing tablet having extended release of active compound over several hours, more precisely a tablet comprising a co-mixture of polyvinyl alcohols (PVAs) and microcrystalline celluloses (MCCs), as characterised above. Surprisingly, it has been found that corresponding active compound-containing tablets have delayed releases of active compound of at least 2 hours, preferably over at least 6 hours, particularly preferably of at least 8 hours, especially preferably of at least 10 hours, and very particularly preferably of at least 12 hours, depending on the active compound employed and on the mixing ratio of the polyvinyl alcohols and microcrystalline celluloses.

In particular, it has been found that active compound-containing tablets which comprise a corresponding directly compressible composition in the form of a co-mixture in an amount of 1-99% by weight, preferably in an amount of 5-95% by weight, very particularly preferably in an amount of 10-90% by weight, based on the total weight of the tablet, have the desired, extended release of active compound. Tablets having particularly high tablet hardnesses which require surprisingly low ejection forces in the production process can advantageously be obtained with such compositions, even on use of low pressing forces. Even on use of a pressing force of 19.5 kN, it is possible to obtain tablets having a tablet hardness of 295.7 N which only require an ejection force of about 66.7 N. In addition, these tablets have only low friabilities of less than 1% by weight, preferably less than 0.5% by weight in particular less than 0.1% by weight, as can be shown by suitable experiments.

Tablets having delayed release of active compound which comprise active compounds from BCS class I, either alone or in combination with other active compounds, can be produced particularly well by compression using the co-mixtures described.

If there is a clinical necessity, however, active compounds from other BCS classes can also be converted into directly compressible administration forms having retarded release of active compound by means of the process according to the invention.

The object of the invention is furthermore achieved by a process for the preparation of directly compressible compositions having extended release of active compound which comprise a co-mixture of microcrystalline celluloses (MCCs) and polyvinyl alcohols (PVAs) in which polyvinyl alcohol is ground to give a fine-grained powder and sieved through an 800 μm sieve and mixed intensively with microcrystalline cellulose (MCCs) having an average particle size dᵥ₅₀ in the range from 60 to 250 μm, and a bulk density in the range from 0.22 to 0.38 g/cm³.

### DETAILED DESCRIPTION OF THE INVENTION

Adequate efficacy of medicaments frequently depends on uniform dosing and requires multiple administration per day so that clinically adequate effect levels in the blood can be obtained over an extended time or undesired side effects can be avoided. However, this multiple administration over the day is not desirable with respect to patient compliance. For the administration of certain active compounds, it is therefore desirable to be able to provide tablet formulations by means of which release of active compound proceeds slowly over hours, so that, when taken regularly, a substantially constant effective blood level becomes established over the day, but it is only necessary to take once per day.

The demands made of the respective composition vary depending on the active compounds to be employed. Depending on their chemical and physical properties, other active compound excipients and tableting aids have to be used, since not every active compound is compatible with every tableting aid or can be processed with one another owing to the chemical and physical properties.

The bioavailability of active compounds can be classified in accordance with a Biopharmaceutics Classification System (BCS), which was developed by Gordon Amidon in the USA in the mid-1990s and has now become part of both a US FDA (Food And Drug Administration) guideline and also a European Medicines Agency guideline for assessment of the bioequivalence of medicaments.

For example, active compounds in BCS class I are active compounds having high solubility and high permeability. Their resorption is controlled only by the speed of stomach and intestine emptying. In the case of active compounds which belong to this class, but whose efficacy is desired over the entire day, attempts are being made to develop formulations which enable delayed, uniform release of active compound.

The Biopharmaceutics Classification System (BCS for short) describes correlations which play an important role in the oral administration of drugs. It is based on the paper by G. Amidon and colleagues from 1995. In this paper, the authors describe that the oral bioavailability of drugs is influenced principally by their solubility, the dissolution rate and the permeability through biological membranes (Amidon G L, Lennernas H, Shah V P, Crison J R. A theoretical basis for a biopharmaceutic drug classification: the correlation of in vitro drug product dissolution and in vivo bioavailability. Pharm Res. 1995; 12:413.)

In the case of active compounds in BCS class 1, both the solubility and the permeability are high.

This means that, if both the solubility and also the permeability of the drug are high, it can be assumed that the absorption rate is determined principally by the rate of stomach and intestine emptying.

Since August 2000, the BCS system has been used in the approval process for proprietary medicinal products of the American approval authority for medicines, the FDA (Food And Drug Administration). Under certain prerequisites, bioavailability and bioequivalence studies can be waived in the application for approval of proprietary medicinal products (PMPs) if it is demonstrated using the BCS system that the new proprietary medicinal product and a PMP which has already been approved for the same drug must be bioequivalent. An application can then be made for a waiver of the obligation to carry out these expensive and in this case unnecessary bioavailability studies. To this end, the drug in the respective medicinal form must meet certain requirements with respect to the principal parameters solubility, permeability and dissolution rate.

Solubility:

The highest dose of the drug must dissolve completely in a maximum of 250 ml of an aqueous dissolution medium in a pH range between pH 1 and pH 7.5.

Permeability:

A drug has high permeability if at least 90% of an administered dose is absorbed by the body. This must be demonstrated by suitable data (for example mass balance studies).

Dissolution Rate:

The medicinal form must ensure rapid release of the drug. This must be demonstrated by suitable in vitro release tests (either rotating basket or rotating paddle method). At least 85% of the corresponding dose must be released within 30 minutes in three different release media (0.1 N HCL, pH 4.5 buffer and pH 6.8 buffer).

A solution to the problem of making a highly soluble active compound available uniformly over hours appears possible here through the use of polymeric active compound excipients, where the latter slowly form a gel in the presence of physiological fluids, such as saliva or gastric or intestinal juice, and release the active compound from the tablet matrix slowly and in a controlled manner by diffusion.

A solution is provided here by polyvinyl alcohols (PVAs), which, as synthetic polymers, are water-soluble resins and have excellent film-forming and emulsifying properties and form a gel in aqueous solutions. According to USP, PVAs are characterised by the formula

(C₂H₄O)ₙ

in which
n denotes an integer in the range from 500 to 5,000. Depending on the molecular size of these polymers and their chemical composition, their properties vary greatly, in particular with respect to the water solubility, but also in relation to the tabletability.

PVAs are prepared from polyvinyl acetate, with some or all of the functional acetate groups being hydrolysed in order to obtain functional alcohol groups. The solubility of the polymer in aqueous media increases with the degree of hydrolysis, but the crystallinity and melting point of the polymer also increases. In addition, the glass transition temperature varies depending on the degree of hydrolysis.

For example, a 38% hydrolysed material does not have a melting point, but has a glass transition temperature of about 48° C., whereas a 75% hydrolysed material has a melting point of about 178° C., an 88% hydrolysed material has a melting point of about 196° C. and a 99% hydrolysed material has a melting point of about 220° C., but the polymer tends to decompose rapidly at a temperature above 200° C.

For the preparation of the compositions according to the invention, use can be made of polyvinyl alcohols (PVAs) of grades 18-88, 26-88 and 40-88 and all grades in between, including grade 28-99 in accordance with JPE or Ph. Eur.

Although polyvinyl alcohols are soluble in water, they are virtually insoluble in almost all organic solvents, with the exception of a few solvents, such as, for example, in ethanol with low solubility. These properties of the polymers make it very difficult to prepare tablet formulations which comprise a high proportion of PVAs and which are directly tabletable.

For use in pharmaceutical formulations, polyvinyl alcohols of different degrees of hydrolysis are specified in the various pharmacopoeia.

The European Pharmacopoeia prescribes that a permissible polyvinyl alcohol for use in pharmaceutical doses must have an ester value of not greater than 280 and an average relative molecular weight between 20,000 and 150,000. The percentage of hydrolysis (H) can be calculated from the following equation:

H=((100−(0.1535)(EV))/(100−(0.0749)(EV)))×100

where EV corresponds to the ester value of the polymer. The ester value means the quantity of potassium hydroxide in mg required to saponify the esters in 1 g of sample. The ester value is calculated from the difference between the saponification value and the acid value.

Thus, according to the monograph in the European Pharmacopoeia, only PVA polymers having a percentage hydrolysis of greater than 72.2% can be employed.

According to the United States Pharmacopeia, polyvinyl alcohols which are suitable for use in pharmaceutical administration forms must have a percentage degree of hydrolysis of between 85 and 89% and a degree of polymerisation of 500 to 5,000. The degree of polymerisation (DM) is calculated by the equation:

DM=(molar mass)/((86)−(0.42(the degree of hydrolysis)))

A PVA which can be employed in pharmaceutical formulations in accordance with the European Pharmacopoeia monograph is a PVA having a degree of hydrolysis of between 72.2% and 90%, which covers both PVAs in accordance with the Ph. Eur. (hydrolysis of more than 72.2%, but less than 90%, and also those in accordance with the USP (degree of hydrolysis between 85-89%). These PVA grades have a molecular weight in the range from 14,000 g/mol to 250,000 g/mol.

As has already been described above, polyvinyl alcohols having a correspondingly high degree of hydrolysis are only directly tabletable under particular conditions, i.e. granulation steps have to be carried out in advance or the PVAs employed must be mixed with further tableting aids and easily compressible binders, so that the proportion of polyvinyl alcohol in the composition as a whole is reduced.

Experiments have now shown that it is not only the degree of hydrolysis of the polyvinyl alcohols employed, and thus the crystallinity, that plays a role for good processability in tablet formulations, but also the physical properties and appearance forms of the commercially available PVA grades employed.

Surprisingly, it has been found that the particle size of the PVA grades used apparently has an influence on the tabletability. In this connection, it has furthermore been found that, depending on the average particle size of the PVA powders, directly tabletable mixtures can be prepared in which the content of PVAs can be more than 60%.

A solution to this problem thus consists in combining a commercially available, pulverulent polyvinyl alcohol in a suitable manner with a very readily compressible component, giving a directly compressible, pulverulent product which predominantly consists of the PVA employed. Consequently, it is possible to produce tablets by simple mixing of the product according to the invention with a desired active compound without further treatment and compression with a suitable pressure. If desired, a few further additives, such as, for example, lubricants and other additives, can be added before the compression of the mixture. An essential feature is that no further treatment is required in order to be able to compress the powder mixture obtained to give tablets.

Surprisingly, experiments have shown that a very wide variety of polyvinyl alcohols can be converted into a directly compressible tableting matrix if microcrystalline celluloses (MCC) are added to ground, pulverulent PVAs. It has been found to be particularly surprising in this connection that very apparently only the MCCs are suitable for achieving direct compression properties of this type; other excipients which usually promote direct compression, such as, for example, directly compressible calcium hydrogenphosphates, including Fujicalin®, which is per se very readily directly tabletable, directly compressible sorbitols (for example Parteck® SI 400), directly compressible mannitols (for example Parteck® M200) or directly compressible starches (for example starch 1500), do not exhibit this effect in combination with PVAs and do not result in directly compressible powder mixtures with the PVAs, as our own investigations have shown.

This effect which has surprisingly been found enables the pharmaceutical formulation scientist now to be provided with a directly compressible premix, predominantly consisting of PVA, for the production of tablets which results in acceleration of a development process of a new tablet formulation.

Microcrystalline cellulose (MCC) is a tableting aid in the preparation of pharmaceuticals and is preferably employed as active compound excipient and is a component for virtually any type of oral dosage forms, such as tablets, capsules, sachets, granules and others.

In pure form, microcrystalline cellulose (MCC) having the general formula (C₆H₁₀O₅)ₙ is white, free-flowing cellulose in powder form which is commercially available with various particle sizes. In pharmaceutical grade, it meets the standards of the usual pharmacopoeias, such as, for example, Ph. Eur., USP/NF or JP. Microcrystalline cellulose serves, inter alia, as indigestible, non-resorbable ballast substance for calorie-reduced foods, for example salad dressings, desserts and ice creams, as release agent or as excipient. As stated in the above description, it is used in pharmacy as a binder or excipient for the production of tablets. In this connection, it has proven suitable for direct tableting and results in hard tablets which have short disintegration times given suitable formulation. MCC is obtained from woody plant parts (not from waste paper). Plant cellulose is freed from non-crystalline cellulose components using dilute hydrochloric acid at temperatures above 100° C. This means that pharmaceutical grade MCC can be obtained by partial hydrolysis of highly pure cellulose and subsequent purification and drying. The hydrolysis can optionally be followed by carboxylation in order to improve the hydrophilic properties.

MCC is insoluble in water, alcohols and organic solvents. In water, MCC forms a three-dimensional matrix consisting of innumerable, insoluble microcrystals, which form a stable thixotropic gel. The advantageous properties of MCC are also retained in the case of temperature-induced changes in the phase state, for example on transition into the frozen state or on heating to elevated temperatures, meaning that MCC is highly suitable for ready mixes for further processing.

The commercially available grades which have average particle sizes Dᵥ₅₀ in the range from 60 to 250 μm, preferably in the range from 80 to 200 μm, particularly preferably in the range from 80 to 150 μm, very particularly preferably in the range from 90 to 140 μm, determined by laser diffraction determination, have proven to be suitable MCCs for achieving adequate tablet hardnesses. MCC grades of this type preferably have bulk densities in the range from 0.22 to 0.38 g/cm³, preferably in the range from 0.24 to 0.35 g/cm³, particularly preferably in the range from 0.28 to 0.33 g/cm³. Suitable commercially available MCC grades which meet these criteria and are qualified for use in pharmaceutical formulations are, for example,

Vivapur 102 (dried in a stream of air, average particle size of about 100 μm, determined by laser diffraction, bulk density 0.28-0.33 g/cm³), Avicel PH 102 (average particle size about 100 μm, bulk density 0.28-0.33 g/cm³) and Emcocel 90M (spray-dried, average particle size of about 100 μm, determined by laser diffraction, bulk density 0.25-0.37 g/cm³).

However, other commercial products not mentioned here which meet the requirements described can also be used in accordance with the invention described here.

It is particularly surprising that addition of suitable microcrystalline celluloses to a very wide variety of PVA grades, in particular to PVAs having a very wide variety of viscosities, gives directly compressible mixtures which predominantly consist of PVAs.

It has proven particularly advantageous for the ratio of the PVAs and MCCs described in the compositions according to the invention to be in the range 2:1 to 1:2, based on the weight, preferably in a ratio in the range from 2:1 to 1:1. Such co-mixtures have proven particularly suitable for the production of tablets having delayed release of active compound. After intensive mixing, the co-mixtures found here of PVA with MCCs have bulk densities in the range 0.43 0.45 g/ml with tapped densities in the range 0.58-0.60 g/ml.

The advantageous properties described of the combinations of PVA and MCC provide the formulation scientist in the pharmaceutical industry, but also in the food industry or in other technical areas, with a material which significantly simplifies the development effort for solid compressed administration forms having extended release of active compound. He needs only mix his active compound to be retarded with the PVA/MCC combination, optionally add a few assistants, in particular lubricants, and then compress this mixture in a tableting machine. The particularly good tableting properties of this matrix have also facilitated the development of retard tablets with active compounds which per se are actually not regarded as directly compressible and had to be granulated in processes carried out in a conventional manner. The use of the PVA/MCC matrix saves development time, investment in equipment and results in improved process reliability in development and production.

An advantageous side effect arises on use of the co-mixtures according to the invention in the tableting process, which consists in that the mixtures according to the invention result in comparatively low ejection forces, enabling significantly smaller amounts of lubricants to be used than is otherwise usual in tableting. Thus, instead of the usual addition of 1% of magnesium stearate, only about a quarter of this amount is required, in some cases even less. Under particular conditions, the addition of such lubricants can also be omitted entirely. This causes an additional improvement in the interparticular binding forces, i.e. harder tablets are obtained for the same pressing force, enabling reproducible release of active compound to be achieved. The latter is due to the fact that the release is essentially controlled via the PVA content, and the reduced addition of hydrophobic magnesium stearate only exerts a slight influence on the release behaviour.

Furthermore, the present invention relates to a process for influencing the tableting properties of fine-grained PVA grades, in particular of ground PVAs, which have per se only low compressibilities. Experiments have shown that these PVAs can be converted into a directly compressible form by combination with MCCs.

Fine-grained PVAs are particularly suitable as retardation matrices, since they can generally be processed very well in order to prepare more homogeneous mixtures with the active compound to be retarded. The latter is of particular importance for the single dosage accuracy “content uniformity” in order always to obtain the same amount of active compound in each individual tablet.

In addition, this type of formulation with fine-grained PVA grades has the advantage that the large surface areas of the fine PVA particles results in more homogeneous gel layer formation after moistening in the gastrointestinal tract, which, when the tablets have been taken by the patient, results in more reproducible and possibly also extended diffusion of the active compound through this gel.

Procedure

For the preparation of the co-mixtures according to the invention, suitable finely ground polyvinyl alcohols (PVAs) are mixed intensively with microcrystalline celluloses (MCCs) and thus converted into co-mixtures which are eminently suitable as directly compressible tableting matrices. This is particularly surprising since blends of such PVAs with other directly tabletable assistants—also very readily compressible per se—on the market do not exhibit this direct compression effect with the pulverulent PVAs. In the following experiments, it can be shown, with reference to a formulation with pulverulent ascorbic acid as model active compound, that PVA/MCC co-mixtures prepared in this way are very highly suitable for the direct compression of poorly compressible active compounds. Furthermore, it can be shown with the tablets produced which comprise corresponding co-mixtures as active compound excipient, that the active compound can be released in a controlled manner over a very long time from tablets produced in this way. The experiments carried out have shown that corresponding active compound-containing tablets have delayed releases of active compound of at least 2 hours, preferably over at least 6 hours, particularly preferably of at least 8 hours, especially preferably of at least 10 hours, and very particularly preferably of up to 12 hours, depending on the active compound employed and on the mixing ratio of the polyvinyl alcohols and the microcrystalline celluloses.

Since the term “directly compressible” is not defined in a binding manner in connection with the preparation of tablet formulations, the pressing behaviour of a commercial very readily directly compressible mannitol (Parteck® M 200 (mannitol)), suitable for use as excipient EMPROVE® exp Ph Eur, BP, JP, USP, E 421, Article No. 1.00419, Merck KGaA, Darmstadt, Germany) is used in the present description as standard for comparison. The aim is to come as close as possible to the behaviour of Parteck® M 200 with respect to its compressibility by means of the directly compressible co-mixtures which comprise PVAs in relatively large amount.

The experiments carried out have shown that active compound-containing tablets which comprise a composition according to the invention in the form of a co-mixture in an amount of 1-99% by weight, preferably in an amount of 5-95% by weight, very particularly preferably in an amount of 10-90% by weight, based on the total weight of the tablet, have the desired, extended release of active compound. Tablets having particularly high tablet hardnesses which require surprisingly low ejection forces in the production process can advantageously be obtained with such compositions as desired even on use of low pressing forces. As has been shown by experiments for the production of placebos, tablets having a tablet hardness of 295.7 N which only require an ejection force of about 66.7 N are obtained even on use of a pressing force of 19.5 kN. In addition, these tablets have only low friabilities, as can be shown by suitable experiments.

The present invention thus provides a process for the preparation of directly compressible compositions having extended release of active compound, giving a co-mixture of microcrystalline celluloses (MCCs) and polyvinyl alcohols (PVAs) in which polyvinyl alcohol is ground to give a fine-grained powder having an average particle size Dᵥ₅₀ in the range from 50 to 260 μm, a bulk density in the range from 0.55 to 0.62 g/ml, a tapped density in the range from 0.72 to 0.85 g/ml and an angle of repose in the range from 35 to 38° and is sieved through an 800 μm sieve, and the powder obtained is mixed intensively with microcrystalline cellulose (MCCs) having an average particle size Dᵥ₅₀ in the range from 60 to 250 μm, and a bulk density in the range from 0.22 to 0.38 g/cm³. In this way, a directly compressible co-mixture having a bulk density in the range from 0.40 to 0.48 g/ml, a tapped density in the range from 0.55 to 0.63 g/ml and an angle of repose in the range from 35 to 38° is obtained, to which various active compounds can be added if desired and which can be compressed to give tablets having delayed release of active compound.

The examples given below disclose methods and conditions for the preparation of PVA/MCC co-mixtures according to the invention. It is self-evident to the person skilled in the art in the area that other methods for grinding and mixing the starting substances than described here are also available.

The examples demonstrate the particular advantages of these PVA/MCC combinations compared with the inadequate compressibilities obtained by PVA combinations with other excipients—but ones which are regarded as particularly readily tabletable.

On blending a PVA/MCC matrix according to the invention with a pulverulent ascorbic acid (as model active compound) which is poorly compressible per se and addition of a very small amount of magnesium stearate as lubricant, tablets of adequate hardnesses with low mechanical abrasion can be obtained by simple direct tableting and are readily available for further treatment, for example for packaging in blister packs or for break-free removal from these blister packs by the patient. Corresponding ascorbic acid-containing tablets show that extended release of ascorbic acid from such PVA/MCC matrix tablets over several hours can be guaranteed.

### LIST OF FIGURES

FIG. 1: FIG. 1 shows a graph of the pressing force/hardness profiles according to the data from Table 1.

FIG. 2: FIG. 2 shows a graph of the pressing force/abrasion profiles according to the data from Table 1.

FIG. 3: FIG. 3 shows the pressing force/hardness profiles of the data of the compositions from Table 4.

FIG. 4: FIG. 4 shows a graph of the pressing force/abrasion profiles using the data from Table 4.

FIG. 5: FIG. 5 shows a graph of the release of ascorbic acid from retard tablets according to sample 1, characterised by data from Table 9.

FIG. 6: FIG. 6 shows a graph of the release of ascorbic acid from retard tablets (sample 2) characterised by data from Table 9.

### EXAMPLES

The present description enables the person skilled in the art to apply the invention comprehensively. Even without further comments, it is therefore assumed that a person skilled in the art will be able to utilise the above description in the broadest scope.

If anything is unclear, it goes without saying that the publications and patent literature cited should be consulted. Accordingly, these documents are regarded as part of the disclosure content of the present description.

For better understanding of the invention and in order to illustrate it, examples are given below which are within the scope of protection of the present invention. These examples also serve to illustrate possible variants. Owing to the general validity of the inventive principle described, however, the examples are not suitable for reducing the scope of protection of the present application to these alone.

Furthermore, it goes without saying to the person skilled in the art that, both in the examples given and also in the remainder of the description, the component amounts present in the compositions always only add up to 100% by weight or mol-%, based on the composition as a whole, and cannot exceed this, even if higher values could arise from the percent ranges indicated. Unless indicated otherwise, % data are thus regarded as % by weight or mol-%, with the exception of ratios, which are reproduced in volume figures.

The temperatures given in the examples and the description as well as in the claims are in ° C.

Equipment/Methods for Characterisation of the Substance Properties

1. Bulk density: in accordance with DIN EN ISO 60: 1999 (German version)
quoted in “g/ml”

2. Tapped density: in accordance with DIN EN ISO 787-11: 1995 (German version)
quoted in “g/ml”

3. Angle of repose: in accordance with DIN ISO 4324: 1983 (German version)
quoted in “degrees”

4. Surface area determined in accordance with BET: evaluation and procedure in accordance with the literature “BET Surface Area by Nitrogen Absorption” by S. Brunauer et al. (Journal of American Chemical Society, 60, 9, 1983) instrument: ASAP 2420 Micromeritics Instrument Corporation (USA); nitrogen; sample weight: about 3.0000 g; heating: 50° C. (5 h); heating rate 3 K/min; quoting of the arithmetic mean from three determinations

5. Particle size determination by laser diffraction with dry dispersal: Mastersizer 2000 with Scirocco 2000 dispersion unit (Malvern Instruments Ltd. UK), determinations at a counterpressure of 1 and 2 bar; Fraunhofer evaluation; dispersant RI: 1.000, obscuration limits: 0.0-10.0%, tray type: general purpose, background time: 7500 msec, measurement time: 7500 msec, procedure in accordance with ISO 13320-1 and the information in the technical manual and specifications from the instrument manufacturer; result given in % by vol.

6. Particle size determination by laser diffraction with wet dispersal: Mastersizer 2000 with Hydro 2000SM wet-dispersion unit (Malvern Instruments Ltd., UK); dispersion medium low-viscority silicone oil (manufacturer: Evonik Goldschmidt GmbH, Germany; manufacturer's name: Tegiloxan3, manufacturer's article no.: 9000305); dispersant RI: 1.403; stirrer speed: 2500 rpm; tray type: general purpose; background time: 7500 msec; measurement time: 7500 msec; obscuration limits: 7.0-13.0%;

procedure in accordance with ISO 13320-1 and the information in the technical manual and specifications from the instrument manufacturer; result given in % by vol.

Procedure: the suspension cell is filled with the low-viscosity silicone oil, the sample is added in portions until the target obscuration range (7.0-13.0%) has been reached, and the measurement is started after a waiting time of 2 minutes.

7. Particle size determination by dry sieving via a sieve tower: Retsch AS 200 control, Retsch (Germany); amount of substance: about 110.00 g; sieving time: 30 minutes; amplitude intensity: 1 mm; interval: 5 seconds; analytical sieve with metal-wire fabric in accordance with DIN ISO 3310; mesh widths (in μm): 710, 600, 500, 400, 355, 300, 250, 200, 150, 100, 75, 50, 32; amount distribution per sieve fraction indicated in the tables as “% by weight of the sample weight”:

8. The tableting tests are carried out as follows:

The mixtures in accordance with the compositions indicated in the experimental part are mixed for 5 minutes in a sealed stainless-steel container (capacity: about 2 l, height: about 19.5 cm, diameter: about 12 cm outside dimension) in a laboratory tumble mixer (Turbula T2A, Willy A. Bachofen, Switzerland).

The magnesium stearate employed is Parteck LUB MST (vegetable magnesium stearate) EMPROVE exp Ph Eur, BP, JP, NF, FCC Article No. 1.00663 (Merck KGaA, Germany) which has been passed through a 250 μm sieve.

The compression to give 500 mg tablets (11 mm punch, round, flat, with bevel edge) is carried out in a Korsch EK 0-DMS instrumented eccentric tableting machine (Korsch, Germany) with the Catman 5.0 evaluation system (Hottinger Baldwin Messtechnik—HBM, Germany).

Depending on the pressing force tested (nominal settings: ˜5, ˜10, ˜20 and ˜30 kN; the effectively measured actual pressing forces are indicated in the examples), at least 100 tablets are produced for evaluation of the pressing data and the pharmaceutical formulation characteristic numbers.

Tablet Hardnesses, Diameters and Heights:

Erweka Multicheck 5.1 (Erweka, Germany); average data (arithmetic means) from in each case 20 tablet measurements per pressing force. The measurements are carried out one day after the tablet production.

Tablet Abrasion:

TA420 friability tester (Erweka, Germany); instrument parameters and performance of the measurements in accordance with Ph. Eur. 7th Edition “Friability of Uncoated Tablets”. The measurements are carried out one day after tablet production.

Tablet Weight:

Multicheck 5.1 (Erweka, Germany) with Sartorius CPA 64 balance (Sartorius, Germany). Quoting of the average value (arithmetic mean) from the weighing of 20 tablets per pressing force. The measurements are carried out one day after tablet production.

9. Ascorbic acid release test: The ascorbic acid-containing compressed tablets (from the compressions with a pressing force of 20 kN) are measured in a SOTAX (Allschwil, Switzerland) in-vitro release apparatus using the “Apparatus 2 (Paddle Apparatus)” described in USP 36 under <711> and under the conditions described therein for “Extended-release dosage forms” (USP=United States Pharmacopoeia). The sampling is carried out automatically via a hose pump system with subsequent measurement in an 8453 spectrometer (Agilent Technologies, USA) and a flow cell.

The averaged values are obtained from the release tests of in each case 6 ascorbic acid-containing tablets pressed with a pressing force of 20 kN.

Ascorbic acid used for tableting: L(+)-ascorbic acid, Ph Eur, USP, NF, Product 83568.290 (VW R, Germany)

Measurement Apparatuses and Measurement Parameters:

1. —Sotax AT7s release apparatus fitted with Apparatus 2 (Paddle Apparatus in accordance with USP 36)
Temperature: 37° C.+/−0.5° C.
Paddle speed: 100 rpm
Volume of release medium per measurement vessel: 900 ml
Tablet weight: 500 mg
Total run time of the measurement: 720 min. (with sampling after 15, 30, 45, 60, 120, 180, 240, 300, 360, 420, 480, 540, 600, 660, 720 min.)

2. —Hose pump for sampling: Sotax CY 7-50 (SOTAX, Switzerland)

3. —8453 spectrometer (Agilent Technologies, USA)
Measurement at 244 nm in a 2 mm flow measurement cell
Evaluation via Excel
Medium preparation: Dosa Prep X8 (DOSATEC GmbH, Germany)

Composition (in % by Weight) of the Release Medium:

| | |
|---|---|
| Potassium dihydrogenphosphate | 0.68% |
| (Article No. 1.04873, Merck KGaA Darmstadt, Germany) | |
| Titriplex III | 0.02% |
| (Article No. 1.08418, Merck KGaA, Darmstadt, Germany) | |
| 85% phosphoric acid | 0.20% |
| (Article No. 1.00573, Merck KGaA Darmstadt, Germany) | |
| demineralised water | 99.10% |

### Characterisation of the Materials Used

1. PVA Grades Used and their Properties:

1.1 Raw Materials for Grinding

1.1.1. PVA 4-88: polyvinyl alcohol 4-88, suitable for use as excipient EMPROVE® exp Ph Eur, USP, JPE, Article No. 1.41350, Merck KGaA, Darmstadt, Germany

1.1.2. PVA 18-88: polyvinyl alcohol 18-88, suitable for use as excipient EMPROVE® exp Ph Eur, USP, JPE, Article No. 1.41355, Merck KGaA, Darmstadt, Germany

1.1.3. PVA 26-88: polyvinyl alcohol 26-88, suitable for use as excipient EMPROVE® exp Ph Eur, USP, JPE, Article No. 1.41352, Merck KGaA, Darmstadt, Germany

1.1.4. PVA 40-88: polyvinyl alcohol 40-88, suitable for use as excipient EMPROVE® exp Ph Eur, USP, JPE, Article No. 1.41353, Merck KGaA, Darmstadt, Germany

1.1.5. PVA 28-99: polyvinyl alcohol 28-99, suitable for use as excipient EMPROVE® exp JPE, Article No. 1.41356, Merck KGaA, Darmstadt, Germany

These PVA grades are in the form of coarse particles with a size of several millimetres which cannot be employed in this form as a directly compressible tableting matrix.

The coarse particles do not allow reproducible filling of the dies and thus also do not allow a constant tablet weight at the high rotational speeds of the (rotary) tableting machines. In addition, only fine-grained PVAs are able to ensure homogeneous distribution of the active compound in the tablet without the occurrence of separation effects; this is absolutely necessary for ensuring individual dosage accuracy of the active compound (content uniformity) in each tablet produced. In addition, only a fine-grained PVA can also ensure the homogeneous gel formation throughout the tablet body that is necessary for reproducible retardation.

For these reasons, the above-mentioned coarse-grained PVA grades must be comminuted, i.e. ground, before use as directly compressible retardation matrices.

1.2 Ground PVA Grades 1.2.1. Ground PVA 4-88, from polyvinyl alcohol 4-88 Article No. 1.41350, Merck KGaA, Darmstadt, Germany

1.2.2. Ground PVA 18-88, from polyvinyl alcohol 18-88 Article No. 1.41355, Merck KGaA, Darmstadt, Germany

1.2.3. Ground PVA 26-88, from polyvinyl alcohol 26-88 Article No. 1.41352, Merck KGaA, Darmstadt, Germany

1.2.4. Ground PVA 40-88, from polyvinyl alcohol 40-88 Article No. 1.41353, Merck KGaA, Darmstadt, Germany

1.2.5. Ground PVA 28-99, from polyvinyl alcohol 28-99 Article No. 1.41356, Merck KGaA, Darmstadt, Germany

Grinding:

The grinding of the PVA grades is carried out in an Aeroplex 200 AS spiral jet mill from Hosokawa Alpine, Augsburg, Germany, under liquid nitrogen as cold grinding in a temperature range from 0° C. to minus 30° C.,

The resultant product properties of the ground PVA grades, in particular the powder characteristics, such as bulk density, tapped density, angle of repose, BET surface area, BET pore volume and the particle size distributions, are evident from the following tables:

Bulk Density, Tapped Density, Angle of Repose, BET Surface Area, BET Pore Volume:

(details on the measurement method, see under Methods)

| | | | Angle | | BET |
|---|---|---|---|---|---|
| | Bulk | Tapped | of | BET | pore |
| | density | density | repose | surface area | volume |
| Sample | (g/ml) | (g/ml) | (°) | (m²/g) | (cm³/g) |
| PVA | 0.61 | 0.82 | 35.1 | 0.1308 | 0.0008 |
| 4-88* | | | | | |
| PVA | 0.57 | 0.76 | 35.5 | 0.1831 | 0.0011 |
| 18-88* | | | | | |
| PVA | 0.56 | 0.74 | 35.5 | 0.2045 | 0.0013 |
| 26-88* | | | | | |
| PVA | 0.59 | 0.77 | 36.9 | 0.1123 | 0.0009 |
| 40-88* | | | | | |
| PVA | 0.58 | 0.76 | 37.7 | 0.2210 | 0.0016 |
| 28-99* | | | | | |

*ground PVA

Particle Distribution Determined by Laser Diffraction with Dry Dispersal (1 Bar Counterpressure):

Figures in μm (details on the measurement method, see under Methods)

| Sample | | | | | | | | |
|---|---|---|---|---|---|---|---|---|
| PVA | Dv5 | Dv10 | Dv20 | Dv25 | Dv30 | Dv50 | Dv75 | Dv90 |
| 4-88* | 21.36 | 33.93 | 60.39 | 75.25 | 91.61 | 177.74 | 380.57 | 790.37 |
| 18-88* | 29.67 | 44.93 | 73.95 | 89.11 | 105.22 | 185.49 | 375.88 | 755.84 |
| 26-88* | 27.76 | 42.32 | 73.01 | 90.14 | 108.67 | 198.51 | 382.65 | 676.96 |
| 40-88* | 31.84 | 50.64 | 89.13 | 109.77 | 131.45 | 230.52 | 413.71 | 634.59 |
| 28-99* | 24.87 | 39.81 | 72.81 | 90.72 | 109.31 | 191.42 | 343.54 | 561.23 |

*ground PVA

Particle Distribution Determined by Laser Diffraction with Dry Dispersal (2 Bar Counterpressure):

Figures in μm (details on the measurement method, see under Methods)

| Sample | | | | | | | | |
|---|---|---|---|---|---|---|---|---|
| PVA | Dv5 | Dv10 | Dv20 | Dv25 | Dv30 | Dv50 | Dv75 | Dv90 |
| 4-88* | 19.09 | 30.21 | 52.69 | 64.83 | 77.87 | 143.83 | 279.64 | 451.94 |
| 18-88* | 26.90 | 40.38 | 65.30 | 78.08 | 91.55 | 159.10 | 321.46 | 607.64 |
| 26-88* | 24.59 | 36.93 | 61.67 | 75.05 | 89.33 | 157.79 | 286.17 | 434.23 |
| 40-88* | 31.03 | 49.47 | 88.54 | 110.06 | 132.79 | 235.87 | 430.35 | 686.10 |
| 28-99* | 24.27 | 39.63 | 74.31 | 93.13 | 112.51 | 196.45 | 350.21 | 570.12 |

*ground PVA

Particle Distribution Determined by Laser Diffraction with Dry Dispersal (3 Bar Counterpressure):

Figures in μm (details on the measurement method, see under Methods)

| Sample | | | | | | | | |
|---|---|---|---|---|---|---|---|---|
| PVA | Dv5 | Dv10 | Dv20 | Dv25 | Dv30 | Dv50 | Dv75 | Dv90 |
| 4-88* | 18.35 | 29.27 | 51.25 | 63.09 | 75.77 | 139.46 | 269.80 | 425.62 |
| 18-88* | 24.55 | 36.60 | 57.91 | 68.48 | 79.45 | 132.37 | 246.56 | 393.59 |
| 26-88* | 25.17 | 38.18 | 64.35 | 78.47 | 93.57 | 167.41 | 317.16 | 514.18 |
| 40-88* | 32.81 | 53.33 | 96.27 | 119.61 | 144.21 | 256.31 | 463.67 | 717.76 |
| 28-99* | 22.33 | 35.92 | 65.94 | 82.31 | 99.37 | 174.84 | 305.50 | 454.03 |

*ground PVA

Particle Distribution Determined by Laser Diffraction with Wet Dispersal (in Low-viscosity Silicone Oil):

Figures in μm (details on the measurement method, see under Methods)

| Sample | | | | | | | | |
|---|---|---|---|---|---|---|---|---|
| PVA | Dv5 | Dv10 | Dv20 | Dv25 | Dv30 | Dv50 | Dv75 | Dv90 |
| 4-88* | 10.03 | 20.10 | 38.02 | 47.82 | 58.31 | 110.91 | 231.64 | 390.95 |
| 18-88* | 17.19 | 30.25 | 50.06 | 59.22 | 68.47 | 111.89 | 212.70 | 357.70 |
| 26-88* | 15.42 | 26.76 | 45.50 | 54.83 | 64.47 | 110.50 | 212.91 | 353.68 |
| 40-88* | 20.41 | 34.80 | 60.35 | 73.32 | 86.96 | 154.96 | 299.57 | 490.08 |
| 28-99* | 14.68 | 25.96 | 47.49 | 58.88 | 70.80 | 127.68 | 240.70 | 376.70 |

*ground PVA

Particle Distribution Determined Via Tower Sieving:

Figures in % by weight (details on the measurement method, see under Methods)

| Sample | | | | | | | | |
|---|---|---|---|---|---|---|---|---|
| PVA | <32 μm | 32-50 μm | 50-75 μm | 75-100 μm | 100-150 μm | 150-200 μm | 200-250 μm | 250-300 μm |
| 4-88* | 3.3 | 7.9 | 12.6 | 12.2 | 19.6 | 12.9 | 10.5 | 6.5 |
| 18-88* | 0.5 | 8.1 | 12.8 | 13.6 | 20.4 | 15.0 | 9.4 | 5.8 |
| 26-88* | 5.3 | 8.4 | 12.3 | 13.6 | 21.8 | 13.1 | 9.0 | 5.0 |
| 40-88* | 2.6 | 5.5 | 8.1 | 8.8 | 17.8 | 14.0 | 10.7 | 7.5 |
| 28-99* | 5.0 | 7.1 | 9.1 | 9.8 | 20.4 | 13.2 | 11.7 | 7.9 |

*ground PVA

| Sample | | | | | | |
|---|---|---|---|---|---|---|
| PVA | 300-355 μm | 355-400 μm | 400-500 μm | 500-600 μm | 600-710 μm | >710 μm |
| 4-88* | 4.5 | 2.8 | 3.5 | 2.0 | 0.9 | 0.8 |
| 18-88* | 4.2 | 2.6 | 3.5 | 2.1 | 1.0 | 1.0 |
| 26-88* | 3.7 | 2.2 | 2.7 | 1.8 | 0.6 | 0.5 |
| 40-88* | 6.6 | 3.9 | 5.9 | 4.1 | 1.9 | 2.6 |
| 28-99* | 5.3 | 3.2 | 3.7 | 2.0 | 0.8 | 0.8 |

*ground PVA

2. Directly Compressible Excipients for the Preparation of the Blends with Polyvinyl Alcohols (Ground)

2.1 Parteck® SI 150 (sorbitol), suitable for use as excipient EMPROVE® exp Ph Eur, BP, JP, JSFA, NF, E 420, Article No. 1.03583, Merck KGaA, Darmstadt, Germany

2.2 Parteck® M 200 (mannitol), suitable for use as excipient EMPROVE® exp Ph Eur, BP, JP, USP, E 421, Article No. 1.00419, Merck KGaA, Darmstadt, Germany

2.3 Parteck® Mg DC (magnesium hydroxide carbonate), heavy, suitable for use as excipient EMPROVE® exp Ph Eur, BP, USP, E 504, Article No. 1.02440, Merck KGaA, Darmstadt, Germany

2.4 Fujicalin®, calcium hydrogen phosphate, anhydrous, DCPA, USP/NF, EP, JP (Fuji Chemical Industry Co, Ltd, Japan, purchased via SEPPIC GmbH, Cologne, Germany)

2.5 Lactose monohydrate (milk sugar), special product for tableting, suitable for use as excipient EMPROVE® exp Ph Eur, BP, NF, JP, Article No. 1.08195, Merck KGaA, Darmstadt, Germany

2.6 Starch 1500® (pregelatinised maize starch) USP/NF, Ph Eur, JPE, IN 516247, Colorcon Limited, UK

2.7 Vivapur® 102 Premium, MCC (microcrystalline cellulose) Ph Eur, NF, JP, JRS Pharma, Rosenberg, Germany

2.8 Avicel® PH 102, MCC (microcrystalline cellulose) Ph Eur, NF, JP, FMC BioPolymer, USA

2.9 Emcocel® 90 M, MCC (microcrystalline cellulose) Ph Eur, NF, JP, JRS Pharma, Rosenberg, Germany

3. Pulverulent Ascorbic Acid (Used as Model Active Compound)

L(+)-Ascorbic acid, Ph Eur, USP, NF, Prod, 83568.290, batch: 11D180012, VWR, Germany

Particle Distribution Determined by Laser Diffraction with Dry Dispersal with 1 Bar Counterpressure:

Figures μm (details on the measurement method, see under Methods)

| Sample | Dv5 | Dv10 | Dv20 | Dv25 | Dv30 |
|---|---|---|---|---|---|
| Ascorbic | 27.63 | 57.03 | 103.64 | 123.02 | 141.50 |
| acid | | | | | |

| Sample | Dv50 | Dv75 | Dv90 | Dv95 |
|---|---|---|---|---|
| Ascorbic | 215.48 | 335.67 | 467.13 | 552.17 |
| acid | | | | |

Particle Distribution Determined Via Laser Diffraction with Dry Dispersal with 2 Bar Counterpressure:

Figures μm (details on the measurement method, see under Methods)

| Sample | Dv5 | Dv10 | Dv20 | Dv25 | Dv30 |
|---|---|---|---|---|---|
| Ascorbic | 24.74 | 52.40 | 100.25 | 120.64 | 140.02 |
| acid | | | | | |

| Sample | Dv50 | Dv75 | Dv90 | Dv95 |
|---|---|---|---|---|
| Ascorbic | 217.41 | 346.52 | 505.33 | 634.51 |
| acid | | | | |

Particle Distribution Determined Via Laser Diffraction with Dry Dispersal with 3 Bar Counterpressure:

Figures μm (details on the measurement method, see under Methods)

| Sample | Dv5 | Dv10 | Dv20 | Dv25 | Dv30 |
|---|---|---|---|---|---|
| Ascorbic | 11.85 | 24.55 | 62.66 | 82.02 | 100.81 |
| acid | | | | | |

| Sample | Dv50 | Dv75 | Dv90 | Dv95 |
|---|---|---|---|---|
| Ascorbic | 177.57 | 304.33 | 451.03 | 558.34 |
| acid | | | | |

Procedure:

1. Compression of the ground polyvinyl alcohols without any additives

2. Preparation of the blends consisting of the various commercial directly compressible excipients with the ground PVA grade 26-88

3. Compression of these blends and tablet characterisation

4. Preparation descriptions of the co-mixtures of ground PVA 26-88 or 40-88 with the microcrystalline cellulose Vivapur® 102

5. Preparation description of the blends of the two co-mixtures obtained under 4. with pulverulent ascorbic acid

6. Compression of these blends and tablet characterisation

7. Testing of the delayed in-vitro release of ascorbic acid from pressed tablets obtained in this way

A) Experimental Results:

1. Compression of the Ground PVAs without any Additives

The ground PVA grades 4-88, 18-88, 26-88, 40-88 and 28-99 are compressed without further additives (also no lubricant) in a Korsch EK 0-DMS tableting machine. Before the compression, the ground PVA grades are passed through an 800 μm hand sieve (diameter 20 cm; Retsch, Haan, Germany) in order to eliminate any agglomerated PVA particles.

Parteck® M200 blended with 1% of Parteck® LUB MST serves as comparison. Note: compression of Parteck® M200 without any lubricant is not possible owing to the resultant very high ejection forces.

TABLE 1
Tableting data of ground PVAs without additives

| | A | | | | | | |
|---|---|---|---|---|---|---|---|
| Sample | Nominal | Actual | B | C | D | E | F |
| PVA 4-88* | 5 | 5.0 | 17.0 | 470.3 | 5.9 | 59.94 | 237.0 |
| | 10 | 10.1 | 40.8 | 491.8 | 5.6 | 8.94 | 383.5 |
| | 20 | 20.7 | 137.2 | 503.2 | 5.1 | 0.35 | 378.3 |
| | 30 | 30.3 | 194.1 | 504.5 | 5.0 | 0.05 | 322.5 |
| PVA 18-88* | 5 | 5.6 | <10 | 409.7 | 5.9 | 100 | 246.4 |
| | 10 | 10.1 | 23.0 | 493.7 | 5.7 | 18.90 | 354.4 |
| | 20 | 19.9 | 89.1 | 499.9 | 5.2 | 1.03 | 382.7 |
| | 30 | 29.9 | 151.1 | 504.0 | 5.0 | 0.14 | 355.7 |
| PVA 26-88* | 5 | 7.3 | 23.9 | 444.7 | 5.6 | 23.37 | 318.2 |
| | 10 | 10.7 | 51.1 | 488.8 | 5.4 | 4.98 | 345.7 |
| | 20 | 19.2 | 129.5 | 492.9 | 5.0 | 0.46 | 327.7 |
| | 30 | 30.7 | 191.8 | 490.9 | 4.8 | 0.06 | 275.7 |
| PVA 40-88* | 5 | 7.6 | 20.5 | 443.1 | 5.7 | 39.93 | 296.7 |
| | 10 | 10.1 | 33.0 | 490.3 | 5.6 | 9.67 | 321.7 |
| | 20 | 18.8 | 150.8 | 506.6 | 5.0 | 0.65 | 317.7 |
| | 30 | 28.5 | 151.4 | 504.6 | 5.0 | 0.12 | 282.9 |
| PVA 28-99* | 5 | 4.7 | <10 | 450.6 | 5.9 | 100 | 169.0 |
| | 10 | 9.7 | 25.5 | 483.9 | 5.5 | 14.22 | 279.5 |
| | 20 | 19.5 | 102.0 | 471.3 | 4.8 | 0.83 | 292.3 |
| | 30 | 30.3 | 178.0 | 472.1 | 4.6 | 0.10 | 263.2 |
| Parteck ® | 5 | 5.2 | 84.1 | 497.8 | 5.1 | 0.21 | 155.8 |
| M200 | 10 | 10.7 | 196.5 | 500.6 | 4.6 | 0.17 | 306.0 |
| | 20 | 20.3 | 340.0 | 499.4 | 4.2 | 0.15 | 513.6 |
| | 30 | 30.0 | 396.7 | 498.3 | 4.0 | 0.16 | 647.6 |

*ground PVA
Key:
A: Pressing force [kN]
B: Tablet hardness after 1 day [N]
C: Tablet weight [mg]
D: Tablet height [mm]
E: Abrasion [%]
F: Ejection force (N)

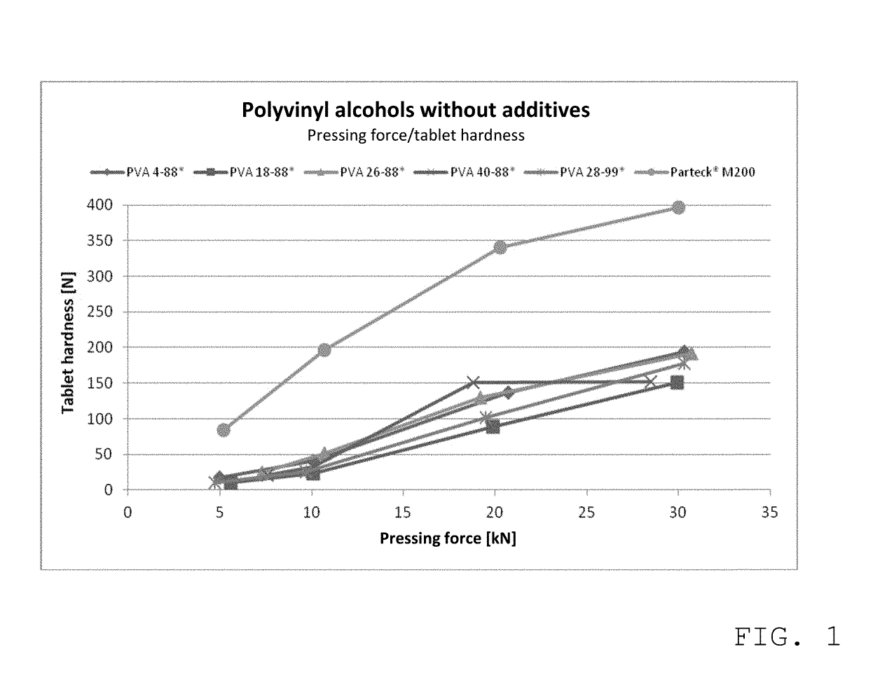

FIG. 1 shows a graph of the pressing force/hardness profiles in accordance with the data from Table 1.

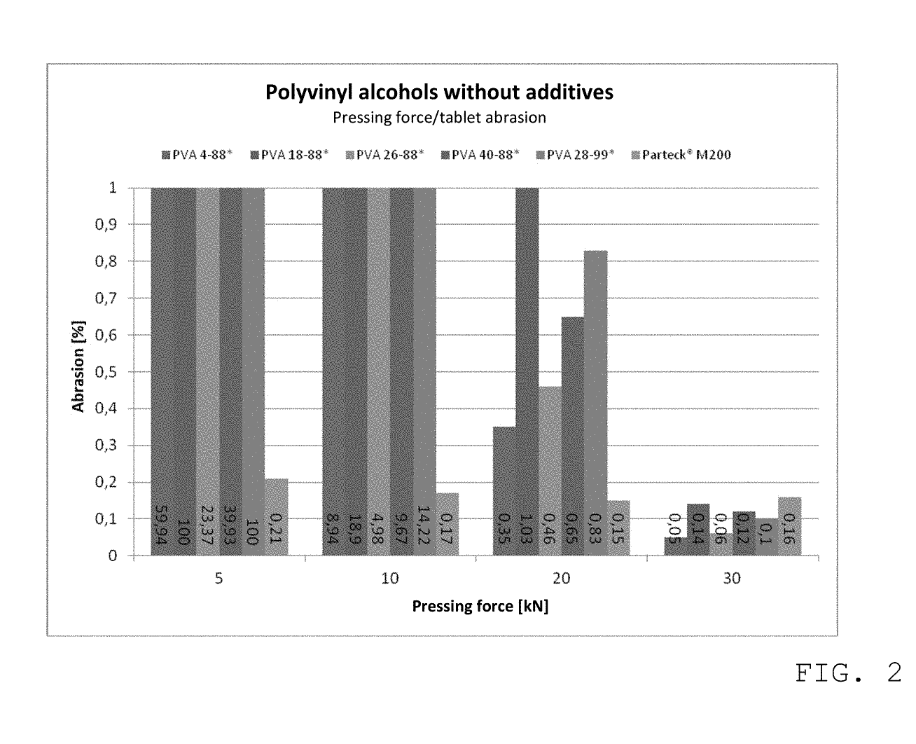

FIG. 2 shows a graph of the pressing force/abrasion profiles in accordance with the data from Table 1.

Result:

a) direct compression of the ground PVA grades is not possible, since tablets of inadequate hardnesses which do not allow safe handling (inadequate pressing force/hardness profiles) are obtained.

b) the tablet abrasion, in particular on use of low pressing forces, is very high.

c) relatively low ejection forces (“self-lubrication effect”) of the ground PVAs; theoretical advantage: stronger interparticular binding forces in the tablet; in the case of the PVAs tested, however, this effect is not sufficient to obtain tablets having adequate hardnesses and low abrasion.

2. Preparation of the Blends of the Directly Compressible Excipients with the Ground PVA Grade 26-88

General description: ground PVA 26-88 is passed through an 800 μm hand sieve. 300 g of this sieved product are weighed out into a 2 l Turbula mixing vessel, 300 g of the corresponding excipient from A to I (see Table 2) are added, and the mixture is mixed for 5 min. in a T2A Turbula mixer.

TABLE 2
Composition of Examples A-C and Comparisons D-I

| | | 50% by weight of |
|---|---|---|
| Composition | 50% by weight of PVA | excipient |
| Example A | PVA 26-88* | Vivapur ^{®}102 |
| Example B | PVA 26-88* | Avicel ^{®} PH 102 |
| Example C | PVA 26-88* | Emcocel ^{®} 90 M |
| Comparison D | PVA 26-88* | Parteck ^{®} SI 150 |
| Comparison E | PVA 26-88* | Parteck ^{®} M 200 |
| Comparison F | PVA 26-88* | Parteck ^{®} Mg DC |
| Comparison G | PVA 26-88* | Fujicalin ^{®} |
| Comparison H | PVA 26-88* | Lactose |
| Comparison I | PVA 26-88* | Starch ^{®} 1500 |

*ground PVA

TABLE 3
Bulk density, tapped density and angle of repose of Examples A-C

| | | | Angle of |
|---|---|---|---|
| | Bulk density | Tapped density | repose |
| Example A | 0.43 g/ml | 0.58 g/ml | 36.4° |
| Example B | 0.44 g/ml | 0.60 g/ml | 35.3° |
| Example C | 0.45 g/ml | 0.59 g/ml | 35.6° |

3. Compression of these Blends and Tablet Characterisation

General description: 1.25 g of magnesium stearate are added to in each case 498.75 g of the co-mixtures from Examples A-C or Comparisons D-I prepared above in a Turbula mixing vessel, the mixture is mixed again for 5 min. in a T2A Turbula mixer and tableted in a Korsch EK 0-DMS eccentric press.

TABLE 4
Tableting data of the co-mixtures of ground PVA 26-88 with excipients

| | A | | | | | | |
|---|---|---|---|---|---|---|---|
| Sample | Nominal | Actual | B | C | D | E | F |
| Example A | 5 | 5.1 | 76.8 | 498.4 | 5.4 | 0.26 | 91.3 |
| | 10 | 10.2 | 171.4 | 502.1 | 4.8 | 0.05 | 91.8 |
| | 20 | 19.5 | 295.7 | 503.4 | 4.5 | 0 | 66.7 |
| | 30 | 30.0 | 354.5 | 502.5 | 4.4 | 0 | 58.6 |
| Example B | 5 | 4.9 | 70.2 | 501.8 | 5.4 | 0.49 | 85.9 |
| | 10 | 9.6 | 153.1 | 506.1 | 4.9 | 0.16 | 87.3 |
| | 20 | 18.4 | 267.3 | 506.6 | 4.5 | 0.07 | 61.1 |
| | 30 | 28.6 | 325.1 | 506.8 | 4.4 | 0.04 | 52.1 |
| Example C | 5 | 4.9 | 71.0 | 494.2 | 5.5 | 0.39 | 90.9 |
| | 10 | 10.2 | 159.6 | 497.0 | 4.9 | 0.06 | 92.3 |
| | 20 | 20.0 | 273.6 | 496.8 | 4.5 | 0 | 64.8 |
| | 30 | 30.4 | 318.0 | 498.2 | 4.4 | 0 | 57.3 |
| Comparison D | 5 | 5.0 | 31.0 | 498.2 | 5.4 | 3.86 | 66.6 |
| | 10 | 9.9 | 86.0 | 502.8 | 4.9 | 0.37 | 94.1 |
| | 20 | 20.5 | 170.0 | 503.6 | 4.5 | 0.08 | 78.6 |
| | 30 | 30.8 | 188.5 | 503.4 | 4.5 | 0.06 | 64.8 |
| Comparison E | 5 | 5.0 | 17.1 | 493.0 | 5.6 | 17.05 | 84.2 |
| | 10 | 10.0 | 51.5 | 499.8 | 5.1 | 1.12 | 138.8 |
| | 20 | 20.3 | 137.6 | 501.3 | 4.7 | 0.21 | 162.9 |
| | 30 | 29.7 | 178.0 | 500.1 | 4.6 | 0.16 | 150.9 |
| Comparison F | 5 | 6.1 | 22.1 | 482.7 | 5.6 | 7.30 | 107.7 |
| | 10 | 10.2 | 50.1 | 501.4 | 5.2 | 1.28 | 133.2 |
| | 20 | 20.9 | 137.4 | 505.0 | 4.7 | 0.13 | 149.0 |
| | 30 | 31.2 | 224.3 | 501.6 | 4.5 | 0.01 | 144.0 |
| Comparison G | 5 | 5.0 | 22.7 | 492.7 | 4.9 | 9.55 | 121.0 |
| | 10 | 10.3 | 48.6 | 495.0 | 4.5 | 1.42 | 148.5 |
| | 20 | 20.6 | 115.2 | 494.5 | 4.1 | 0.27 | 126.2 |
| | 30 | 29.6 | 161.6 | 492.1 | 3.9 | 0.10 | 102.0 |
| Comparison H | 5 | 4.9 | <10 | 374.7 | 5.1 | n.b. | 57.3 |
| | 10 | 10.2 | 16.7 | 488.2 | 5.0 | 100 | 98.4 |
| | 20 | 19.9 | 50.2 | 495.3 | 4.6 | 2.00 | 127.3 |
| | 30 | 29.0 | 77.4 | 497.3 | 4.5 | 0.50 | 135.1 |
| Comparison I | 5 | 5.0 | <10 | 468.2 | 5.5 | 100 | 54.8 |
| | 10 | 9.8 | 28.9 | 492.3 | 5.1 | 10.49 | 69.1 |
| | 20 | 19.6 | 77.3 | 494.1 | 4.7 | 0.78 | 57.2 |
| | 30 | 30.0 | 98.7 | 494.3 | 4.6 | 0.30 | 50.6 |
| Parteck ® | 5 | 5.2 | 84.1 | 497.8 | 5.1 | 0.21 | 155.8 |
| M200 | 10 | 10.7 | 196.5 | 500.6 | 4.6 | 0.17 | 306.0 |
| | 20 | 20.3 | 340.0 | 499.4 | 4.2 | 0.15 | 513.6 |
| | 30 | 30.3 | 396.7 | 498.3 | 4.0 | 0.16 | 647.6 |

Key:
A: Pressing force [kN]
B: Tablet hardness after 1 day [N]
C: Tablet weight [mg]
D: Tablet height [mm]
E: Abrasion [%]
F: Injection force (N)

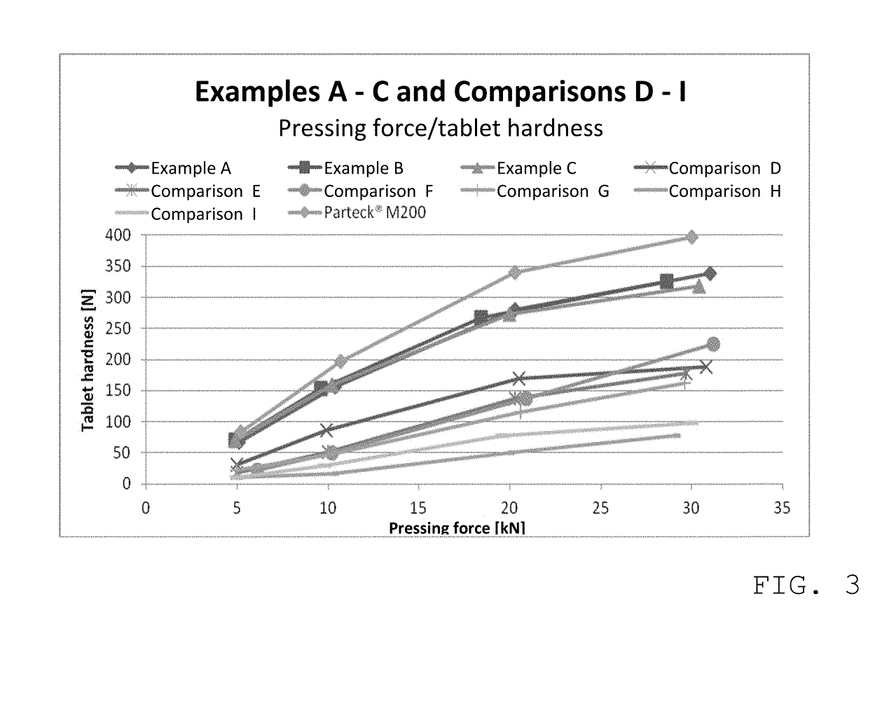

FIG. 3 shows the pressing force/hardness profiles of the data for the compositions from Table 4.

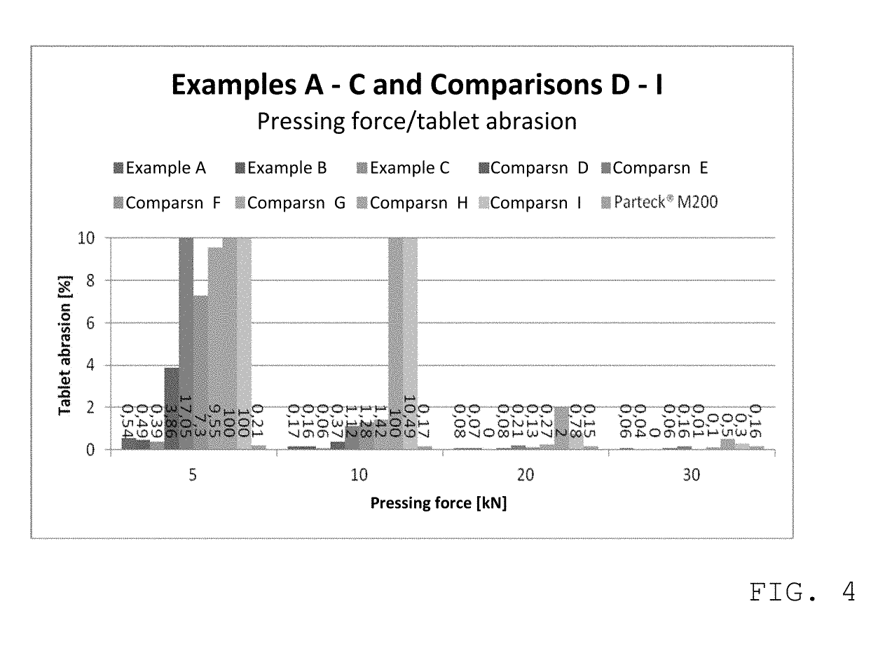

FIG. 4 shows a graph of the pressing force/abrasion profiles with reference to the data from Table 4.

Result:

a) only the co-mixtures based on ground PVA 26-88 with the three MCC grades tested (Examples A-C) give tablets having adequate hardnesses at all 4 pressing forces tested and come very close in their compressibilities to the internal standard Parteck® M 200; all other co-mixtures exhibit significantly lower tablet hardnesses at the same pressing forces

b) tablets based on Examples A-C exhibit a reduced friability compared with the other matrices, in particular at low pressing forces.

4. Preparation Description of the Co-Mixtures of Ground PVA 26-88 and Ground PVA 40-88 with Vivapur® 102

### Example A

Ground PVA 26-88 is passed through an 800 μm hand sieve. 300 g of the sieved product are weighed out into a 2 l Turbula mixing vessel, 300 g of Vivapur® Type 102 are added, and the mixture is mixed for 5 min. in a T2A Turbula mixer.

### Example D

Ground PVA 40-88 is passed through an 800 μm hand sieve. 300 g of the sieved product is weighed out into a 2 l Turbula mixing vessel, 300 g of Vivapur® Type 102 are added, and the mixture is mixed for 5 min. in a T2A Tubula mixer.

TABLE 5
Composition of Examples A and D

| | | 50% by weight of |
|---|---|---|
| Composition | 50% by weight of PVA | excipient |
| Example A | PVA 26-88* | Vivapur ^{®}102 |
| Example D | PVA 40-88* | Vivapur ^{®}102 |

*ground PVA

TABLE 6
Bulk density, tapped density and angle
of repose of Examples A and D

| | | | Angle of |
|---|---|---|---|
| | Bulk density | Tapped density | repose |
| Example A | 0.43 g/ml | 0.58 g/ml | 36.4° |
| Example D | 0.43 g/ml | 0.59 g/ml | 36.3° |

TABLE 7
Tableting data of Example A and Example D

| | A | | | | | | |
|---|---|---|---|---|---|---|---|
| Sample | Nominal | Actual | B | C | D | E | F |
| Example A | 5 | 5.1 | 76.8 | 498.4 | 5.4 | 0.26 | 91.3 |
| | 10 | 10.2 | 171.4 | 502.1 | 4.8 | 0.05 | 91.8 |
| | 20 | 19.5 | 295.7 | 503.4 | 4.5 | 0 | 66.7 |
| | 30 | 30.0 | 354.5 | 502.5 | 4.4 | 0 | 58.6 |
| Example D | 5 | 5.0 | 64.2 | 500.4 | 5.4 | 0.49 | 76 |
| | 10 | 10.3 | 146.9 | 505.7 | 4.9 | 0.15 | 90 |
| | 20 | 20.1 | 247.4 | 506.0 | 4.5 | 0.08 | 62 |
| | 30 | 32.0 | 296.6 | 506.0 | 4.5 | 0.07 | 91 |

Key:
A: Pressing force [kN]
B: Tablet hardness after 1 day [N]
C: Tablet weight [mg]
D: Tablet height [mm]
E: Abrasion [%]
F: Injection force (N)

5. Preparation Description of the Blends of the Two Co-Mixtures Obtained Under 4. with Pulverulent Ascorbic Acid

Sample 1: 150 g of ascorbic acid are added to 450 g of co-mixture Example A and mixed for 5 min. in a T2A Turbula mixer. 1.25 g of magnesium stearate are sieved into 498.75 g of this mixture via a 250 μm sieve, and the mixture is mixed for 5 minutes in a T2A Turbula mixer.

Sample 2: 150 g of ascorbic acid are added to 450 g of co-mixture Example D and mixed for 5 min. in a T2A Turbula mixer. 1.25 g of magnesium stearate are sieved into 498.75 g of this mixture via a 250 μm sieve, and the mixture is mixed for 5 minutes in a T2A Turbula mixer.

6. Compression of Samples 1 and 2 and Tablet Characterisation

TABLE 8
Tableting derivative of samples 1 and 2

| | A | | | | | | |
|---|---|---|---|---|---|---|---|
| Sample | Nominal | Actual | B | C | D | E | F |
| Sample 1 | 5 | 5.3 | 34.8 | 501.6 | 5.1 | 3.19 | 73.4 |
| | 10 | 10.0 | 74.4 | 504.6 | 4.7 | 0.61 | 88.2 |
| | 20 | 20.0 | 140.0 | 504.5 | 4.3 | 0.21 | 88.0 |
| | 30 | 30.5 | 173.7 | 505.2 | 4.2 | 0.14 | 87.6 |
| Sample 2 | 5 | 5.1 | 25.5 | 498.0 | 5.1 | 7.15 | 73.6 |
| | 10 | 11.2 | 61.9 | 501.1 | 4.6 | 0.75 | 95.6 |
| | 20 | 20.8 | 125.8 | 503.5 | 4.4 | 0.12 | 96.0 |
| | 30 | 31.1 | 157.6 | 506.3 | 4.2 | 0.08 | 95.7 |

Key:
A: Pressing force [kN]
B: Tablet hardness after 1 day [N]
C: Tablet weight [mg]
D: Tablet height [mm]
E: Abrasion [%]
F: Injection force (N)

Result:

1. Even in combination with a pulverulent ascorbic acid which is regarded as poorly directly compressible, tablets of adequate hardness and low friability which can be handled without problems are obtained using co-mixtures Example A and D according to the invention; the use of directly compressible ascorbic acid grades which are otherwise usual can thus be omitted.

2. The ejection forces of the mixtures with Example A and Example D are unusually low—even in the case of the only very small amount of added magnesium stearate; this causes lower wear of the punch tools and tableting machines.

3. The relatively small amount of added magnesium stearate means that the target retarded release of active compound is essentially determined only by the amounts and properties of the PVA used; the known interfering influence of the hydrophobic magnesium stearate on the active compound release behaviour is minimised.

7. Testing of the Delayed In-Vitro Release of Ascorbic Acid from Pressed Tablets Obtained in this Way

TABLE 9
Results of the release of ascorbic acid from retard tablets of sample 1
and sample 2 (pressed at a pressing force of 20 kN)
(Figures in % by weight of the amount of ascorbic acid released,
based on the expected total amount of ascorbic acid/tablet,
measurement of 6 tablets per sample)

| | Sample 1 | | | Sample 2 | | |
|---|---|---|---|---|---|---|
| | (tablets pressed | | | (tablets pressed | | |
| | at a pressing | | | at a pressing | | |
| Time | force of 20 kN) | | | force of 20 kN) | | |
| (min.) | Min | Max | Average | Min | Max | Average |
| 0 | 0 | 0 | 0 | 0 | 0 | 0 |
| 15 | 14 | 20 | 17 | 15 | 18 | 16 |
| 30 | 21 | 28 | 24 | 22 | 25 | 24 |
| 45 | 26 | 34 | 30 | 27 | 32 | 30 |
| 60 | 31 | 40 | 35 | 32 | 37 | 35 |
| 120 | 46 | 57 | 51 | 46 | 53 | 50 |
| 180 | 60 | 72 | 65 | 58 | 66 | 63 |
| 240 | 71 | 84 | 77 | 67 | 77 | 73 |
| 300 | 81 | 91 | 86 | 72 | 82 | 78 |
| 360 | 89 | 98 | 93 | 78 | 89 | 85 |
| 420 | 94 | 101 | 97 | 86 | 93 | 90 |
| 480 | 97 | 103 | 100 | 90 | 96 | 94 |
| 540 | 98 | 104 | 101 | 96 | 102 | 99 |
| 600 | 98 | 104 | 101 | 98 | 103 | 101 |
| 660 | 98 | 104 | 101 | 99 | 103 | 101 |
| 720 | 98 | 104 | 101 | 100 | 103 | 102 |

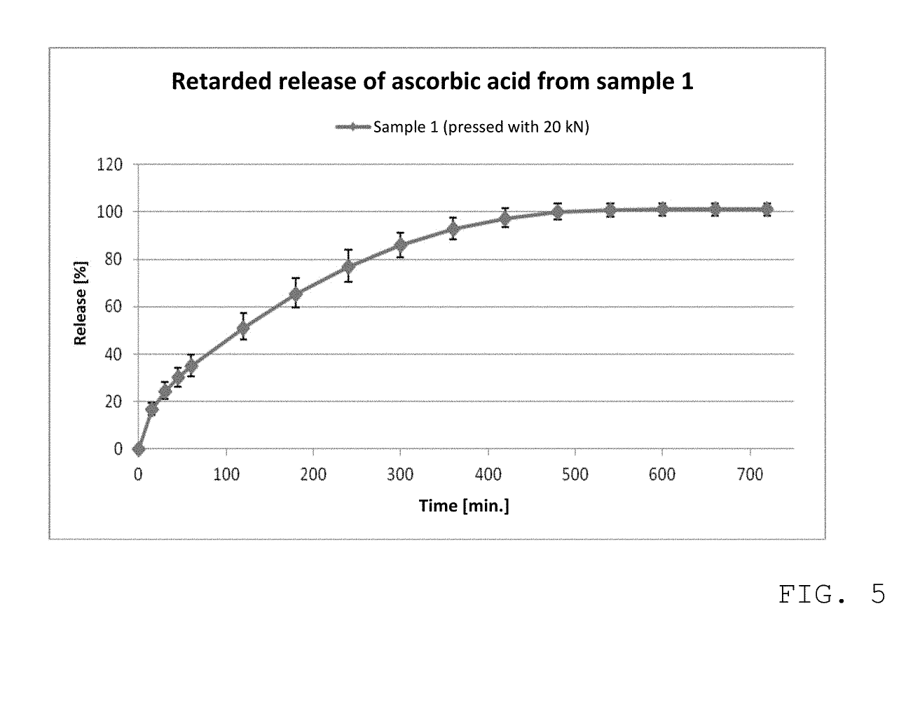

FIG. 5 shows a graph of the release of ascorbic acid from retard tablets in accordance with sample 1, characterised by data from Table 9.

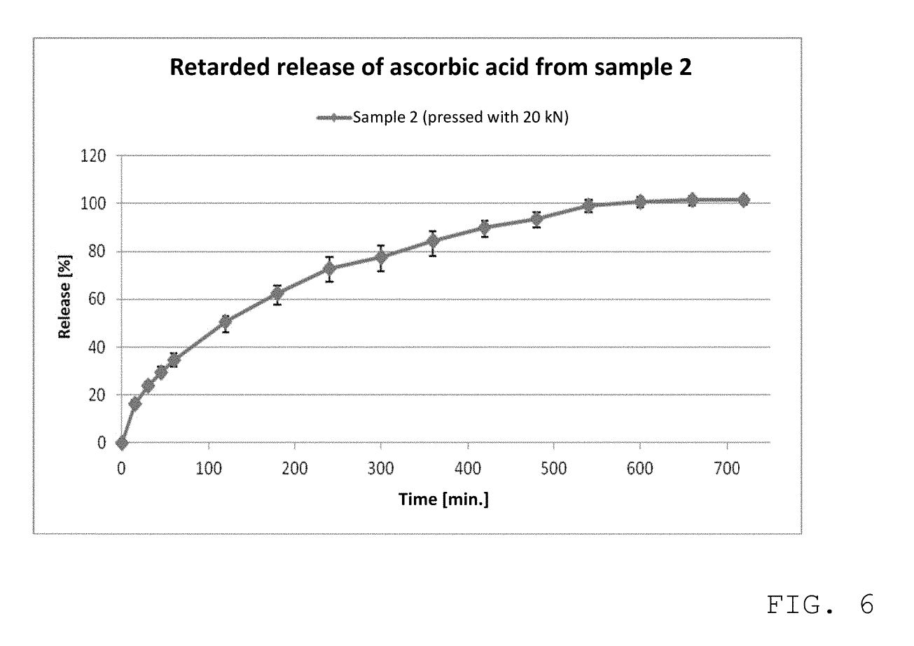

FIG. 6 shows a graph of the release of ascorbic acid from retard tablets in accordance with sample 2 with reference to the data from Table 9.

Result:

retarded in-vitro release of the model active compound ascorbic acid is possible over several hours

B) Conclusion

1. The co-mixtures of ground PVA with MCC result in very readily directly tabletable tablet matrices. Even at relatively low pressing forces, tablets having adequate hardness and mechanical stability can be produced.

2. With these matrices, even active compounds which are per se regarded as poorly tabletable can be converted into tablets having good pharmaceutical formulation properties, in particular with respect to hardness and mechanical stability, in a direct tableting process.

3. With the aid of these matrices, retard tablets having release of active compound lasting over several hours can be produced rapidly and unproblematically by direct tableting.

1. Directly compressible composition having extended release of active compound, comprising a co-mixture of microcrystalline celluloses (MCCs) and polyvinyl alcohols (PVAs).

2. Directly compressible composition according to claim 1, comprising a co-mixture of microcrystalline celluloses (MCCs) and polyvinyl alcohols (PVAs), where the latter meet the requirements of the pharmacopoeias (PhEur, USP or JPE.

3. Directly compressible composition according to claim 1, comprising polyvinyl alcohols (PVAs) of grades 18-88, 26-88 and 40-88 and all grades in between in accordance with the requirements of the pharmacopoeias PhEur, USP or JPE, including grade 28-99 in accordance with the requirements of JPE or PhEur.

4. Directly compressible composition according to claim 1, comprising polyvinyl alcohols (PVAs) which conform to PhEur and which have been obtained by polymerisation of vinyl acetate and by subsequent partial or virtually complete hydrolysis of the polyvinyl acetate.

5. Directly compressible composition according to claim 1, comprising polyvinyl alcohols (PVAs) which have been obtained by 85%-89% hydrolysis.

6. Directly compressible composition according to claim 1, comprising polyvinyl alcohols (PVAs) having an average relative molecular weight in the range between 20,000 and 150,000 g/mol which have a viscosity in accordance with PhEur in the range 3-70 mPa·s, (measured in a 4% solution at 20° C.).

7. Directly compressible composition according to claim 1, comprising polyvinyl alcohols (PVAs) which have an ester value of not greater than 280 mg of KOH/g (degree of hydrolysis >72.2 mol %).

8. Directly compressible composition according to claim 1, which comprises polyvinyl alcohols (PVAs) as water-soluble resin, which is characterised in accordance with USP by the formula
(C₂H₄O)ₙ,
in which
n denotes an integer in the range from 500 to 5,000.

9. Directly compressible composition according to claim 1, comprising PVA and MCC in a co-mixture in a ratio in the range 2:1 to 1:2, preferably in a ratio in the range 2:1 to 1:1.

10. Directly compressible composition according to claim 1, characterised in that the co-mixture of PVA with MCCs have bulk densities in the range 0.40-0.48 g/ml with tapped densities in the range 0.55-0.63 g/ml.

11. Tablet comprising a composition according to claim 1 which result in tablets having a tablet hardness of 295.7 N, even on use of a pressing force of 19.5 kN, and require an ejection force of about 66.7 N.

12. Active compound-containing tablet having extended release of active compound over several hours, comprising a co-mixture of polyvinyl alcohols (PVAs) and microcrystalline celluloses (MCCs) in accordance with claim 1.

13. Active compound-containing tablet having extended release of active compound over several hours, comprising a directly compressible composition in the form of a co-mixture in accordance with claim 1 an amount of 1-99% by weight, preferably in an amount of 5-95% by weight, very particularly preferably in an amount of 10-90% by weight, based on the total weight of the tablet.

14. Active compound-containing tablet according to claim 12, which have particularly high tablet hardnesses, even on use of low pressing forces, and require low ejection forces.

15. Active compound-containing tablet according to claim 12, which exhibit low friabilities of less than 1% by weight, preferably of less than 0.5% by weight, in particular of less than 0.1% by weight.

16. Active compound-containing tablet according to claim 11 having delayed release of active compound of at least 2 hours, preferably over at least 6 hours, particularly preferably of at least 8 hours, especially preferably of at least 10 hours, and very particularly preferably of at least 12 hours.

17. Active compound-containing tablet according to claim 11 having delayed release of active compound, comprising active compounds in BCS class I, either alone or in combination with other active compounds.

18. Process for the preparation of directly compressible compositions according to claim 1 having extended release of active compound, comprising a co-mixture of microcrystalline celluloses (MCCs) and polyvinyl alcohols (PVAs), characterised in that polyvinyl alcohol is ground to give a fine-grained powder and sieved through an 800 μm sieve, and mixed intensively with microcrystalline cellulose (MCCs) having an average particle size Dᵥ₅₀ in the range from 60 to 250 μm, and a bulk density in the range from 0.22 to 0.38 g/cm³.
